(19) United States
(12) Patent Application Publication
(10) Pub. No.: US 2005/0255129 A1
(43) Pub. Date: Nov. 17, 2005

(54) Treatments and methods for treatment, diagnosis and prophylaxis

(76) Inventor: Luc Montagnier, New York, NY (US)

Correspondence Address: MILDE & HOFFBERG, LLP, 10 BANK STREET, SUITE 460, WHITE PLAINS, NY, 10606, US

(21) Appl. No.: 10/513,359

(22) PCT Filed: May 5, 2003
(86) PCT No.: PCT/US03/13993

§ 371 Date: Nov. 2, 2004

(30) Foreign Application Priority Data
May 3, 2002 (US) 60377823

(51) Int. Cl. A61K 39/21

(52) U.S. Cl. 424/208.1

(57) ABSTRACT

HIV may be folmd associated with mycoplasma in some clinical samples, and in in vitro studies. A vaccine and a method of treating or prophylaxing a human infection, comprising administering a vaccine adapted for raising an immune response to mycoplasma associated antigens, and/or HIV associated antigens. The invention also encompasses pharmaceutical treatment, diagnostic tests, and screening methods for treatment of mycoplasma and/or HIV infection.

### FIELD OF THE INVENTION

The present invention relates to the field of pharmaceutical therapies and medical treatments, for HIV infection and related pathologies.

### BACKGROUND OF THE INVENTION

It is well established that human immunodeficiency virus (HIV) plays a significant role in the development of AIDS, a human pathological syndrome characterized by significant defects in cellular immunity, and resulting opportunistic infections. HIV is a type of retrovirus which is well known in the art.

For many years, it has been proposed that mycoplasmas might have involvement in the etiology of AIDS, although the particular role of mycoplasmas has not been elucidated, and treatments directed toward mycoplasma infection were not deemed clinically useful in treating the fundamental pathologies of the disease.

Mycoplasmas are small bacteria devoid of rigid walls and are external, rarely internal parasites of cells of higher organisms. In animal and man, they colonize mucous membranes (oral, genital, bronchioalveolar, intestinal). Only a few species are considered as harmful and can cause serious diseases (pneumonia, abortion in humans). Earlier work by Luc Montagnier and collaborators have associated mycoplasma with HIV infection. A. Blanchard & L. Montagnier. “AIDS Associated Mycoplasmas”, Ann. Rev. Microbiol. 48:687-712 (1994). In particular, antibodies raised against an attachment protein of mycoplasma (adhesin) could, in some instances, neutralize or inhibit the infectiousness of HIV1 and HIV2, but results were variable from one experiment to another. L. Montagnier et al., “Inhibition of Infectiousness of HIV Prototype Strains by Antibodies Raised Against a Peptidic Sequence of Mycoplasma”, C. R. Acad. Sci. Paris 311:425-430 (1990).

It is also known that highly active anti-retrovial therapies (HAART), typically including a combination of reverse transcriptase inhibitors and protease inhibitors, though successful in blocking HIV activity in vitro, do not clear the virus in vivo.

### SUMMARY OF THE INVENTION

The present inventor has discovered that, indeed, mycoplasmas play a significant role in HIV pathology, and that treatment of mycoplasma infection in HIV infected patients is useful.

The present invention therefore provides diagnostic tools designed to detect mycoplasma or related organism infection, pharmacological therapies for controlling mycoplasma infection, and medical therapies for managing AIDS and HIV-related pathologies.

It has been found, according to the present invention, that in coinfection of HIV and mycoplasma, HIV infectiousness and mycoplasma markers are associated with particles having the same density, and presumably are physically bound. This, in conjunction with other evidence that clinical HIV infection may be associated with mycoplasma, results in the conclusion by the inventor that specific treatments and vaccines directed towards mycoplasma and related organisms may play a significant role in the treatment and prevention of HIV infection. The entire disclosure of WO/02089744 is expressly incorporated herein by reference. It is understood that, in accordance with the present invention, mycoplasma and/or mycoplasma like organisms may be involved in HIV disease and HIV infection.

According to one aspect of the present invention, treatments and vaccines for mycoplasma are administered to patients to reduce mycoplasma activity. Preferably, antiviral and/or immunostimulant therapies are coadminstered with the antimycoplasma treatment, such that the immunocompetence of the patient is restored, and the putative mycoplasma reservoir for HIV is reduced. This combination holds promise as an effective treatment, or even cure for the illness associated with chronic HIV infection.

Antioxidant therapies may also hold value, both to boost host defenses, and to particularly suppress HIV and mycoplasma damage to the cells. See, U.S. Pat. No. 6,350,467, U.S. Pat. No. 6,204,248 and U.S. Pat. No. 6,159,500, expressly incorporated herein by reference.

The highly active antiretroviral therapy (HAART) is known to suppress HIV to nearly undetectable levels in the plasma, for example, less than 50 copies per milliliter. However, it is also clear that there remain viral reservoirs within the host which reactivate the infection once the HAART is interrupted. Thus, while HAART may be useful in restoring immune function by reducing the immunosuppressive effect of HIV or HIV proteins, it is insufficient to achieve a cure for the pathologies associated with HIV infection.

One likely reservoir for HIV is mycoplasma. Data available to the inventor indicates that HIV antigens and infectivity are associated with sucrose density peaks at which live mycoplasma bands, about 1.21-1.22. Further, mycoplasma DNA, as determined by PCR using generic mycoplasma primers (e.g. 16S rRNA), as well as specific M. pirum primers, is also found at sucrose density peak associated with HIV particles, about 1.15-1.16. A specific antibody preparation capable of neutralizing M. pirum was able to completely abolish infectivity of HIV associated with the mycoplasma peak. A portion of the peak associated with HIV particles is also neutralizable by the same antibody. This indicates that there is a physical association of the two biological entities. This results in the proposal of new treatments for HIV infection and vaccine-based prevention of HIV transmission, and in particular human-HIV infection therapies, diagnostics, and prophylaxis directed to mycoplasma antigens and organisms.

For example, a vaccine directed toward mycoplasma proteins, e.g., adhesin or other surface proteins, may assist in prevention of transmission of HIV infection to new hosts. A preferable vaccine for prevention of sexual HIV transmission includes, for example, components effective for inducing production of secretory IgAs binding to mycoplasma (or both mycoplasma and HIV). Likewise, in HIV infected hosts, particularly those who are rendered immunocompetent through antiviral treatments and/or immunostimulatory treatments, a mycoplasma vaccine (or vaccine directed toward both mycoplasma and HIV) may provide a useful long-term tool for keeping the infection under control.

In addition to or instead of vaccines, antibiotics and other pharmacological therapies directed toward reducing mycoplasma activity may also be beneficial for the HIV infected patient.

A pharmaceutical formulation may be provided, in oral unit dosage form, for treating human HIV infection, comprising at least one antiretroviral agent, such as a reverse transcriptase inhibitor or protease inhibitor, and at least one antimycoplasma agent, such as a tetracycline-class antibiotic (e.g., doxycycline, ciprofloxacin, azitliromycin, minocycline, clarithromycin and/or Augmentin), each being present in a therapeutic amount, that is, having a benefCcial therapeutic effect, and having compatible dosage regimens. Antioxidants, such as glutathione, may also be incorporated in for formulation, which may have non-specific host immunocompetence enhancing effects and specific antiretroviral or antimycoplasma effects. It may also be possible to treat mycoplasma infection with a mycoplasma-specific phage, similar to Mycoplasma Virus P1, Taxonomy NCIB ID: 35238.

### BRIEF DESCRIPTION OF THE DRAWINGS

The invention is shown by way of example in the drawings, in which:

FIG. 1 shows in vitro results of a representative sucrose gradient showing the bull of HIV viral particles bands at a density of 1.15-1.16, and a second infectious peak at the density of 1.21 to 1.22, representing mycoplasma-associated activity; and

FIG. 2 shows in vivo results from a representative sucrose gradient showing undetectable activity at a density of 1.15-1.16, but infectious activity at a density of 1.21, representing mycoplasma associated activity.

### DETAILED DESCRIPTION OF THE PREFERRED EMBODIMENTS

Mycoplasmas as Vectors of HIV

In Vitro

A cell line (CEM), generally used to grow laboratory strains of HIV was found to be contaminated with two mycoplasma species, M. Arginini and M. Pirum. This was shown both by molecular techniques (PCR) and by neutralization of the mycoplasma outgrowth in acellular medium (SP4) with specific antibodies against these two species. The cells were infected with the LAI strain of HIV1. At the peak1 of virus production, as measured by reverse transcriptase activity and p24 antigen determination in the supernatant medium, the latter was harvested and clarified by centrifugation at 3,000 rpm for 10 minutes in a refrigerated centrifuge. An aliquot of the resulting supenatant was layered on the top of a 5 ml Beckman centrifuge tube and centrifuged for 2 h, at 35,000 rpm in a SW56 Beckman rotor, so that particles with a specific density could reach equilibrium. Controls were evaluated to ensure that indeed large particles, such as viruses or mycoplasmas, had reached equilibrium under these conditions of centrifugation. The time of two hours was chosen to minimize biological inactivation of viral and microbial entities in high sucrose concentrations.

At the end of centrifugation, collection of fractions was carried out from the bottom of the tube, after piercing the latter by a sterile needle. An aliquot of every 5 fractions was taken to measure the refractive index of the sucrose medium with a refractometer in order to deduce the density gradient. These operations were performed in a vertical laminar flow hood under sterile conditions.

Then, the fractions are rapidly diluted ⅓ in rpmi culture medium (with 10% fetal calf serum) and stored at 4° C. until processing. Several parameters are measured in each fraction: p24 antigen or RT to determine where the virus band is located, which was usually at the density 1.15-1.16.

The density of mycoplasma is determined either by previously labeling the cells with 3H uracil, which is specifically incorporated into mycoplasma nucleic acids and not in those of the host CEM cells, or by PCR using primers specific for all mycoplasmas. Usually this density is 1.21, although there are heavier (1.24) and lighter forms (1.16, 1.14).

Each fraction was also tested on activated peripheral blood lymphocytes (PBL) from blood donors, for HIV infectiousness. The production of virus was measured by p24 assay in the supernatant of the infected cells (in 96 well plates) at days 7, 10, 13 after infection. Usually 50 μl of the sucrose gradient fractions were diluted ⅕ in the PBL well (200μ), in order to minimize the toxic effect of the residual sucrose.

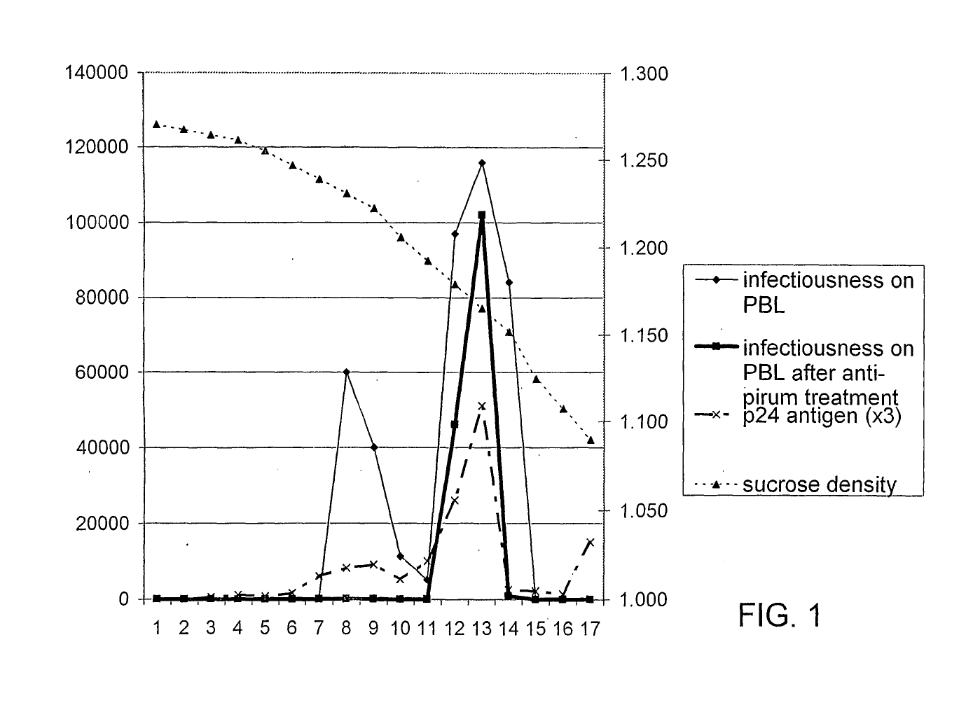

FIG. 1 shows the results of a representative experiment. The bulk of HIV viral particles bands at a density of 1.15-1.16, as evidenced by p24. This peak is also infectious on PBL. However, a second infectious peak can be detected at the density of 1.21 to 1.22. The infectiousness of this peak was abolished by pre-treatment of the gradient fractions with specific anti-M. pirum rabbit antiserum ( 1/100 final dilution for 1 Hr at 37° C.) while that of 1.16 peak was not. However on the light edge of the 1.16 peak, an infectious fraction (density 1.14) was also neutralized by the anti M. pirum serum.

These results indicate that the infectiousness of the HIV 1.21 peak and other accessory peaks, is dependant on Mycoplasma pirum. A supported hypothesis is therefore that HIV particles are coated with mycoplasma material.

It is noted that, in this particular experiment, a shoulder of p24 could be seen at the 1.21 density (about 10% of total viral p24) while 40% of the viral infectiousness is associated with this region. This fraction has a much higher ratio of infectious particles to physical particles than the peak at 1.16, which is the density of free retroviral particles.

In other experiments, this shoulder could not be detected, while retaining high infectiousness. This would indicate (this is also the case of the light fraction No. 14) that the fractions which do not band a 1.16 contain less defective particles than the latter, or enter the susceptible cells by a more efficient mechanism.

It is also of interest that serums which specifically neutralize the other accompanying mycoplasma, M. arginini, have no effect on virus infectiousness, indicating that the association between M. pirum and HIV is specific. However in other experiments, other mycoplasma, such as M. penetrans, also seem to be associated with infectious HIV. These observations are apparently not restricted to in vitro situations, as shown in the following section.

In Vivo

Plasma from HIV infected patients was analyzed in sucrose gradient according to the procedure described above. Fractions were collected and assayed for infectiousness in PBLs.

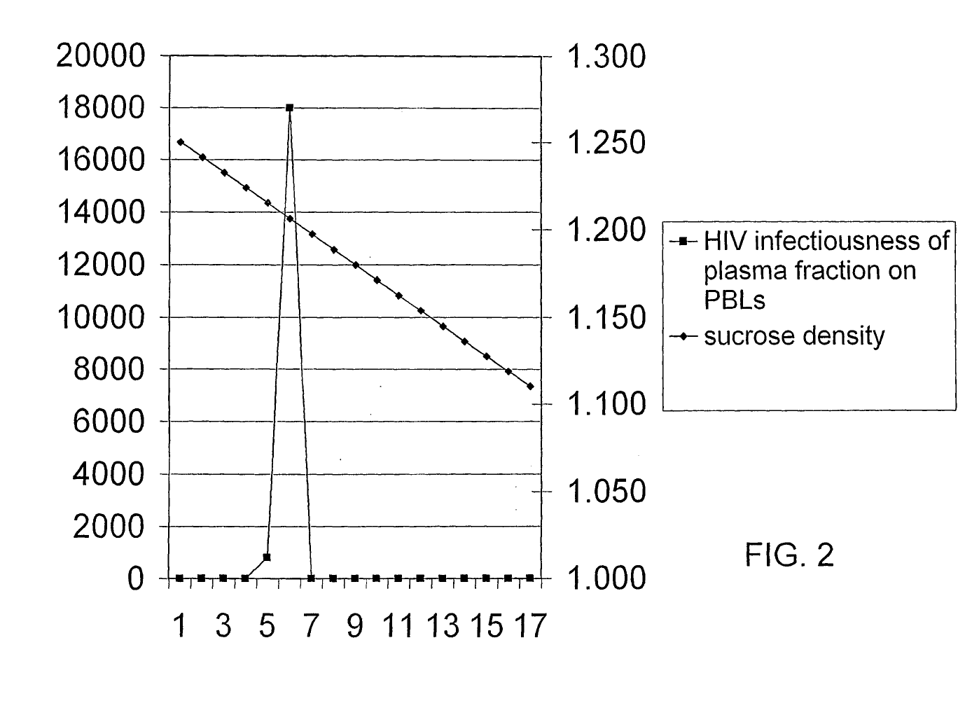

FIG. 2 shows results from a representative experiment. The plasma was taken from a patient infected with HIV, subtype A, and treated by triple antiretroviral therapy. The plasma viral load was undetectable at the time of the experiment (less than 200 RNA copies/ml). No p24 antigen could be detected in any of the fractions of the gradient. However fractions corresponding to the 1.21 density, that of mycoplasma, was infectious on PBLs and the virus recovered from the latter had the classical properties of HIV, i.e. banding at 1.16 upon centrifugation on a second sucrose gradient.

Although the mycoplasma species could not be grown further on PBL or in SP4 medium, and therefore could not be identified, the density of the infectious particles from plasma would suggest also an association with a mycoplasma or a mycoplasma like-organism (MLO).

The results also indicate that minute amounts of infectious virus could still circulate in patients responding well to highly active anti-retroviral therapy (HAART). It is known that a fraction of HIV present in patients resists the effect of antiretroviral drigs, so that the infection cannot be eradicated without also controlling the reservoir(s). It is possible that HIV coating by mycoplasma proteins increases its tropism for long lasting cells where the virus can stay latent for a long time.

In some patients, also treated by HAART, infectious HIV was not present in plasma but could be recovered from the red blood cell fraction, after coculture with PBLs from blood donors. A mycoplasma like organism could be identified in the erythrocyte fraction by metabolic labeling with 3H uracil, but could not be cultivated further and characterized. This organism could be a mycoplasma or MLO organism closely related to M. genitalum, which is known to absorb on erythrocytes. Free HIV particles do not bind to erythrocytes.

The data might indicate that the physical association of HIV with mycoplasma or MLO is not infrequent and may play an important role in HIV propagation and disease.

### EXAMPLE 1

### Immunotherapy

The present research indicates that, if an infectious fraction of HIV is associated with mycoplasma, that no vaccination raised only against HIV proteins will be 100% efficient in preventing transmission by the sexual routes. Therefore, the present invention provides a partial or adjunct vaccine against transmission of HIV based on mycoplasma immunity. Further, in HIV infected patients, a related vaccine or immunotherapy is provided comprising an immunostimulant formulation which results in immunity to mycoplasmas or mycoplasma surface antigens.

For example, a vaccine may be formulated which comprises mycoplasma proteins, either as a main active constituent, or in combination with HIV-active vaccine components.

An antiserum raised against a peptide corresponding to the binding side to the host cell of the adhesin from M. genitalum has previously been found to neutralize infectiousness of HIV1 and HIV2 prototype strains (LAI and ROD). The present data, resulting from testing of a polyclonal anti M. pirum serum, confirms that such antibodies may form an important component of HIV-related vaccines. It is also noted that mycoplasma and MLO may also be associated with other diseases, and for example have been reportedly associated with the so-called “Gulf War Syndrome”, so that mycoplasma and MLO treatments may have use in the prevention and control of various other pathologies, and are not limited to HIV-related pathologies.

According to one aspect of the present invention, a composite vaccine is provided including mycoplasma surface proteins: (in a first step, membrane extracts of several known M. species found in AIDS patients, such as M. pirum, M. penetrans, and M. felmentans); in a second step, purified adhesin proteins, for example provided by genetic engineering, from the same species, could be used. The present inventor has previously sequenced the gene for M. pirum adhesin, J. Bacteriology, 176:781-788 (1994).

In a proposed vaccine formulation, the mycoplasma adhesin will be added to HIV proteins, such as the modified gp160, Nef and Tat, with proper adjuvants. A crude mycoplasma extract may itself serve as adjuvant, as it is a strong T cell activator. A mucosal presentation may be used to induce secretory IgAs.

### EXAMPLE 2

### Antibiotic Therapy

The present invention also provides for the use of antibiotic therapy for treatment of HIV infected patients.

According to experimental data, in vitro experiments revealed rapid appearance of mycoplasma mutants resistant to a single antibiotic. Like HIV, mycoplasmas have a high variability potential, due to several mechanisms including intragenic recombination. Therefore, similarly to HIV, a combination therapy involving several anti-mycoplasma or anti-MLO antibiotics is proposed.

According to a further aspect of the invention, the efficacy of anti-mycoplasma or anti-MLO treatments will be assessed by the disappearance of the 1.21 HIV infectiousness peak in plasma. Therefore, during the course of treatment, which may be a chronic, extended or long-lasting treatment, a patient is periodically monitored for evidence of mycoplasma or HELLO activity. In the event of detection of activity, the regimen of antibiotics may be altered in order to provide continued suppression of activity.

### EXAMPLE 3

### Combination Therapies

Combination therapy for HIV and mycoplasma, together with therapeutic immunization against HIV and mycoplasma proteins may ultimately result in the eradication of HIV infection in previously HIV seropositive individuals.

That is, based on evidence of the concept that mycoplasma or MLOs serve as a reservoir for HIV in the body that is resistant to antiviral therapy, a treatment of both organisms simultaneously may result in an effective “cure” for HIV infection. While it is known that HIV is a retrovius, and therefore integrates into host cell DNA, the host defenses, especially early in the course of the disease, may be sufficient to eradicate infected cells, provided that no reservoir exists which shields the virus and allows reinfection.

Likewise, these same principles may also be applied to other human retroviral diseases or suspected retroviral diseases, or other diseases in which mycoplasmas or MLOs may be cofactors.

### EXAMPLE 4

### Vaccine

A vaccine for use in humans is provided targeted against M. pirum, which, as discussed above, has been implicated in certain HIV infections.

The vaccine comprises a membrane extract of M. pirum grown in a liquid culture medium, for example SP-4. The extract is treated with a non-ionic detergent, to disrupt membranes and release membrane proteins. Affinity chromatography is employed to purify and concentrate the proteins. By using appropriate affinity media, the selectivity of the vaccine may be improved. The membrane proteins are incorporated into liposomes, for administration either systemically or to mucous membranes. The liposome preparation is stored at 4° C. in a stabilizing medium until use.

It should be understood that the preferred embodiments and examples described herein are for illustrative purposes only and are not to be construed as limiting the scope of the present invention, which is properly delineated only in the appended claims.

1. A method for treating a virus-infected human, or providing prophylaxis against viral infection, comprising administering a vaccine adapted for raising an immune response to mycoplasma, or mycoplasma-like organism associated antigens.

2. The method according to claim 1, wherein the antigens comprise mycoplasma adhesin antigens.

3. The method according to claim 1, wherein the antigens comprise mycoplasma surface proteins.

4. The method according to claim 1, wherein said vaccine is further adapted for raising an immune response to a target virus.

5. The method according to claim 4, wherein said target virus is HIV.

6. The method according to claim 1, wherein the human is infected with HIV.

7. (canceled)

8. The vaccine according to claim 15, said vaccine preparation being effective for the treatment or prophylaxis of a human immunodeficiency virus (HIV) or simian immunodeficiency virus (SIV) infection further comprising a component adapted for inducing an immune response to HIV or SIV antigens.

9. The method of claim 1, wherein the human is infected with HIV, further comprising the steps of administering at least one antibiotic agent targeting a mycoplasma metabolic process, and administering an antioxidant in an effective amount to additionally inhibit mycoplasma replication.

10. The method according to claim 9, further comprising the step of administering a treatment to induce an immune response toward HIV.

11. A method for treating HIV infection, comprising detecting an association of HIV with mycoplasma infection, and providing a treatment directed toward reducing mycoplasma infection in dependence on said detected association.

12. The method according to claim 11, wherein the provided treatment is directed specifically toward reducing the detected HIV associated mycoplasma.

13. The method according to claim 11, wherein the HIV infection is treated with antiviral pharmacological agents, and said treatment directed toward reducing mycoplasma infection comprises additionally administering pharmacological agents to reduce mycoplasma activity.

14. The vaccine preparation according to claim 15, wherein said vaccine preparation is specifically adapted for producing a host immune response to M. pirum.

15. A vaccine preparation specifically adapted for producing a host immune response to mycoplasma or mycoplasma-like organisms in primates.

16. A method for determining a treatment for a human retrovirus infection, comprising detecting a presence of mycoplasma, determining a sensitivity of mycoplasma to a proposed treatment, and selecting a treatment based on said determining.

17. A method of screening a drug for activity against HIV-mycoplasma complex, comprising isolating a HIV-mycoplasma complex, and testing the infectiousness of the complex on human lymphocytes, wherein an effect of a drug to be screened is determined at either the formation of the complex or the infectiousness of the complex.

18. The method according to claim 17, wherein said isolating comprises separating the HIV-mycoplasma complex based on density.

19. The method according to claim 1, further comprising the steps of administering highly active antiretroviral therapy to reduce viral load to less than about 200 copies of viral RNA per milliliter of plasma; determining that the human has sufficient immune ability to respond to foreign antigens; and thereafter administering a said vaccine.

20. The method according to claim 19, wherein the administered vaccine further is directed toward an HIV antigen.

21. The method according to claim 19, further comprising the step of administering to the human at least one antibiotic effective against mycoplasma.

22. The method according to claim 19, further comprising the step of administering an antioxidant in an effective amount to suppress mycoplasma activity.

23. The method according to claim 22, wherein said antioxidant is administered in an amount based on in vitro suppression of mycoplasma.

24. The method according to claim 22, wherein said antioxidant is administered in an optimal amount to stimulate the human host immune system and suppress mycoplasma activity.

25. The vaccine according to claim 15, wherein said preparation comprises mycoplasma surface proteins or peptides representative of mycoplasma surface proteins, and proteins designed to raise an immune response to HIV.

26. The vaccine according to claim 25, wherein the proteins designed to raise an immune response to HIV comprise modified proteins able to raise immune response against a plurality of strains of HIV.

27. The vaccine according to claim 25, wherein the proteins designed to raise an immune response to HIV comprise modified proteins able to raise cellular and humoral immune response.

28. A pharmaceutical formulation, in oral unit dosage form, for treating a human infected with HIV, comprising at least one antiretroviral agent, and at least one antimycoplasma agent, each being present in a therapeutic amount.
